(19) United States
(12) Patent Application Publication
(10) Pub. No.: US 2004/0185558 A1
(43) Pub. Date: Sep. 23, 2004

(54) Method for culturing brain tissue in three dimensions

(76) Inventors: Corinne Griguer, Birmingham, AL (US); Caroline Cofer, Vestavia Hills, AL (US); Lori McMahon, Vestavia Hills, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/768,316

(22) Filed: Jan. 30, 2004

Related U.S. Application Data
(60) Provisional application No. 60/443,589, filed on Jan. 30, 2003.

(51) Int. Cl. C12N 5/08

(52) U.S. Cl. 435/368

(57) ABSTRACT

A method for culturing 3-dimensional sections of CNS tissue, such as brain tissue, under microgravity conditions is disclosed. In one example, the microgravity conditions are simulated microgravity conditions generated by a culture vessel containing said tissue. Brain tissue cultured under simulated microgravity conditions as described herein remain viable, retain natural tissue architecture and organization and respond to well characterized electrophysiological stimuli in the same manner as brain tissue studied under acute conditions. The culture method described may be used for any brain tissue desired. The brain tissues and cells generated by the method of the instant disclosure may be used for a variety of purposes, including, but not limited to, development of disease models, physiological models, drug screening and drug evaluation.

The present application claims priority to and the benefit of U.S. provisional patent application No. 60/443,589, filed on Jan. 30, 2003. This invention was made with government support under grant NS041382 awarded by the National Institutes of Health. The government has certain rights in the invention.

### FIELD OF DISCLOSURE

The present disclosure relates in general to methods for culturing brain tissues, and specifically to methods for 3-dimensional culture of brain tissues and model systems resulting from such methods.

### BACKGROUND

Previous studies in microgravity conditions conducted during space missions and studies conducted in the terrestrial 1 G environment under simulated microgravity conditions (SMC) have demonstrated the effect of microgravity on cell/tissues functions. For example, lymphocytes and bone tissues have been found to be particularly effected by microgravity. Moreover, changes in mechanical forces applied to cells and tissues, such as are experienced during exposure to microgravity, are involved in altered cellular morphology, such as shape and adhesion, as well as altered rates of proliferation and differentiation.

Changes in central nervous system functioning have been documented after long term exposure to microgravity. These include neurovestibular disturbances, cephalic fluid shifts, alterations in sensory perception, changes in proprioception, psychological disturbances and cognitive changes. Studies regarding these studies have predominantly been carried out using whole animals flown in space and subject to microgravity conditions, and then examined after sacrifice upon return. Animal studies conducted after long term exposure to microgravity have shown altered plasticity of the neural cytoarchitecture, decreased neuronal metabolism in the hypothalamus, and changes in neurotransmitter concentrations. These changes in CNS function at the molecular, cellular and tissue levels are beginning, but are incomplete. The possibility that environmental factors encountered in microgravity can influence molecular, cellular and tissue functions in the CNS suggest that microgravity research on CNS tissues, such as the brain, will yield insight into the ontogeny and function of the cells and tissue comprising the CNS and also provide insight into the performance of astronauts during extended exposure to microgravity conditions.

The nervous system in general and the brain in particular are remarkable for their ability to respond and adapt to environmental changes. For space flights, adaptation occurs both during exposure to microgravity (˜0 G) and, after re-entry, as a result of exposure to the terrestrial environment (1 G). It has been shown that altering the gravitational environment of a subject may lead to anatomical, chemical and physiological changes in the cerebral cortex subserving sensory and motor function (Van der Loos and Woolsey, 1973; Hubel et al., 1977; Kaas et al., 1983; Donoghue, 1995; Buonomano and Merzenich, 1998; Jones, 2000; Sur and Leamey, 2001). Day et al. (1998) reported a decrease in the level of GFAP (glial fibrillary acidic protein) in the brain of adult rats during 14 days of spaceflight.

Recent studies have reported changes in the expression of myosin isoforms in neonatal rats after spaceflight (Adams et al., 2000; Ikemoto et al., 2001). At the cortical level, spaceflight studies have revealed increases in the overall density of dendritic spines in the sensorimotor cortex of adult rats after 7 and 14 days in microgravity (Belichenko, 1988; Belichenko and Krasnov, 1991). Changes in the afferent information that reaches the sensorimotor cortex induced by selected muscle atrophy and the altered use of hindlimb muscles in microgravity may affect cortical synaptic organization (Blue and Parnavelas, 1983; Bähr and Wolff, 1985; Luhmann and Prince, 1991; Agmon and O'Dowd, 1992; Agmon et al., 1993, 1996; Micheva and Beaulieu, 1996; White et al., 1997; DeFelipe et al., 1997; Wells et al., 2000).

Morphological and structural evidence from neurons of the brain indicate that neurons experience hypoactivity during microgravity (Krasnov, 1994). In particular, cellular oxidative potential decreases in dorsal root ganglia (Ishihara et al., 1997), acetylcholin-esterase activity and RNA content decease in cervical ganglia (Roy et al., 1996), and total catecholamine excretion decrease in humans (Robertson et al. 1994). Neurotransmitter receptor changes have also been reported under microgravity conditions.

Most of these studies have been done in the rat cortex, looking at serotonin, dopamine, noradrenergic, and GABA receptors. For example, animal studies have shown altered plasticity of the neural cytoarchitecture, decreased neuronal metabolism in the hypothalamus and change in neurotransmitter concentration (Newberg and Alavi, 1998).

The hippocampus plays a central role in memory consolidation, a process for converting short-term memory into cortically stored, long lasting memory in the mammalian brain. The search for the molecular and cellular mechanisms underlying learning and memory has made significant progress in recent decades. The discovery of long-term potentiation (LTP) marked a beginning for the molecular and cellular exploitation of synaptic plasticity (Bliss, T. V. P. and Collingridge, G. L. 1993). The role of NMDA-receptor-dependent synaptic plasticity in learning and memory has been explored using gene-knockout techniques. These experiments demonstrate that the CA-1-hippocampal NMDA receptor is required for formation of hippocampus-dependent spatial and non-spatial memories (Tsien, J. K. et al. 1996; Cell 87, 1327-1338; Rampon, C. et al. 2000; Nat.Neurosc. 3, 238-244; Shimizu, E. 2000; Science 290, 1170-1174).

In general, however, studies focusing on the effect of microgravity on the brain and other CNS tissues have only reported ultrastructural changes, including loss of axon terminals in the somatosensory, visual, and olfactory cortices (D'yachkova, 1991; D'yachkova and Sluch, 1991), increased ribbon synaptic plasticity in hair cells of the utricular maculas (Ross, 1994), and increased neural adaptation in the primary afferents of the semicircular canal (Correia et al, 1992; Ross, 1994). Very little is known about the cellular and molecular effects associated with the growth of brain tissue under microgravity conditions. This is primarily a function of the lack of a method to grow brain and other CNS tissues under SMC conditions in a 1 G terrestrial environment, and/or the expense of obtaining sufficient quantities of such tissues exposed to microgravity conditions in space. The instant disclosure provides a method to grow and culture brain tissue and other CNS tissue under simulated microgravity conditions. The brain tissue cultured under SMC as described herein remains viable in culture for extended time periods, retains natural architecture and organization and responds to well characterized stimuli in a similar manner to brain tissue examined immediately after removal. In addition, certain portions of the brain tissue cultured under SMC as described herein continue to proliferate.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

FIGS. 1A and B show the cellular organization of tissues grown under simulated microgravity conditions as compared to control hippocampal tissue. FIG. 1A shows Niss'l staining from hippocampal brain tissue grown under simulated microgravity conditions for 10 days and FIG. 1B showing Niss'l staining of control hippocampal tissue taken from the original brain tissue used to initiate the hippocampal cell culture.

FIGS. 2A and B show the results of a viability assay on 10 day old hippocampal cultures grown under simulated microgravity conditions. FIG. 2A shows calcein AM staining of viable hippocampal cells and FIG. 2B shows ethidium homodimer-1 staining of non-viable hippocampal cells.

FIGS. 3A and B show dendritic field EPSP evoked in hippocampal tissues cultured under simulated microgravity conditions and in control brain tissue. FIG. 3A shows field EPSP from hippocampal tissue grown under simulated microgravity conditions, while FIG. 3B shows field EPSP evoked in control acute hippocampal brain slice. Pair pulse stimulation was conducted at 0.1 Hz. Note that the second stimulation evoked a larger response in both conditions indicating the presence of a short-term plasticity characteristic of healthy acute hippocampal slices.

FIGS. 4A-C show the modulation of field EPSP in hippocampal tissue grown under SMC. FIG. 4A shows control field EPSP evoked in regular medium, FIG. 4B shows field EPSP evoked in regular medium supplemented with 1 mM CoCl₂ and FIG. 4C shows field EPSP evoked after washout of the CoCl₂.

FIGS. 5A-C show the effect of glutamatergic antagonists on field EPSP in hippocampal tissues cultured under simulated microgravity conditions. Field EPSP were recorded in hippocampal tissues under control conditions (FIG. 5A, no antagonist added) or in the presence of the specific glutamatergic antagonists DNQX (FIG. 5B) and APV (FIG. 5C). Both 10 μmM DNQX (FIG. 5B) or 100 μM APV (FIG. 5C) are able to partially block field EPSPs evoked in hippocampal tissues grown under simulated microgravity conditions.

FIGS. 6A and B show Western blot analysis of acute hippocampal tissue sections. Whole cell lysates from hippocampal slices were separated on a 4-20% Tris-glycine PAGE gradient gel (Biorad) and probe with antibody. In FIG. 6A, GlyR subunit antigen was visualized using mAb 4a. In FIG. 6B, GFAP protein was detected by MAb36. Positions of size markers (mol. wt/1000) are as indicated. Lanes 1-6 represent proteins extracted from hippocampal slices (400 μm) from half of the brain (6 slices represented) rostral to caudal.

FIGS. 7A and B show immunoblotting of hippocampal tissue (400 μm slice) grown under simulated microgravity conditions. In FIG. 7A, Fibrous astrocytes present in the brain tissue are labeled by a monoclonal antibody against GFAP (MAb36, 1:500). The detection method used an anti-mouse secondary antibody tagged with a fluorescent label (1:400). FIG. 7B is a higher magnification of a portion of the image in FIG. 7A.

### DETAILED DESCRIPTION

Described is a method for culturing CNS tissue, such as brain tissue, under microgravity conditions over prolonged periods of time. In one embodiment, the microgravity conditions are simulated microgravity conditions (SMC) and the SMC are generated by the culture vessel containing the tissue. Throughout this specification, the discussion and examples employ SMC to illustrate the principles of the present disclosure; however, the teachings of the present disclosure would apply to any microgravity condition that was naturally occurring (i.e., such as that occurring the space or the upper atmosphere).

Using the culture method disclosed, natural tissue characteristics (such as, but not limited to, tissue architecture and physiology) are retained. Currently, organotypic cultures or studies using disassociated cells are used to study the function of CNS tissue such as brain tissue. Each of these methods suffers from various limitations. For organotypic brain slice cultures, the natural cellular architecture is quickly altered by mechanical forces acting on the tissue slice and by the interaction of the tissue slice with its substrate. Therefore, natural tissue architecture was retained for a short time period (typically on the order of hours). Since natural architecture and organization was not retained, the experimental results obtained with these types of culture systems may not be relevant. For example, the tissue cultured in this manner is subject to very high surface tensions created by the air-media interface and/or are subject to continued fluid shear stress. Typical tissue slices for organotypic cultures are on the order of 400 μm thick when the culture process is initiated. However, as the culture process is continued, the slice thins to a thickness of around 100 μm in response to mechanical forces acting on the slice and due to interactions of the tissue slice with the substrate provided. As a result, natural tissue architecture is not preserved. Therefore, the complex neuronal circuitry of the tissue is not retained. As a result, the natural tissue physiology is not preserved. The applicants have found that it is very difficult to obtain population recordings from slices grown in organotypic cultures due to the thin nature of the tissue slice. Cultures of disassociated cells have also been employed. However, the use of these systems suffers from the drawback that natural cellular architecture and organization is completely destroyed.

As one example of the difficulties with conventional culture methods for brain tissue, consider the problems associated with studying neural circuits. A neural circuit is the pattern of connections between specific groups of neurons. All functions of the nervous system require that the correct pattern of connections (or neural circuit) remain intact. If part of the circuit is broken due to cell death, loss of a neurotransmitter, etc then function will be lost.

Dissociating neurons and plating them in 2-dimensional culture disrupts the normal pattern of connections. Synaptic connections between neurons will develop in 2-dimensional culture but these connections are made promiscuously between neighboring neurons that might not otherwise be connected in vivo. Since the correct sequence of connections will be disrupted, the normal pattern of activity will be lost.

In 2-dimensional slice cultures, the neurons are not dissociated from their usual synaptic contacts so that the circuit remains intact. This allows for properties of the circuit to be studied under conditions that more closely mimics the function of the intact brain. Even though the tissue grown in 2-D maintains an intact synaptic circuit, it is not as robust as that observed in the natural environment and in tissue grown under SMC (and therefore a 3-dimensional environment) as described herein. As discussed above, in 2-dimensional slice cultures, the tissue is grown under strong surface tension resulting from an air/liquid interface which causes the tissue to flatten and the circuit to loose connections. In the method disclosed, there is no tension on the tissue thus permitting the normal structure of the tissue to be maintained and allowing for a larger number of native synaptic connections to be maintained.

Brain tissue slices cultured in 3-dimensional environment under SMC by the methods of the instant disclosure provide a solution to these issues. The tissues cultured as described herein retain natural tissue characteristics over prolonged periods of time when cultured under SMC as described. Natural tissue characteristics include, but are not limited to, the maintenance of natural: tissue architecture, tissue organization, physiological responses to certain stimuli, physiological response to agonists and inhibitors, electrophysiological responses and maintenance of neural circuits. The natural tissue characteristics may vary depending on the type of tissue cultured. A characteristic of a tissue cultured under SMC as described herein is considered a natural tissue characteristic if the characteristic of the cultured tissue approximates the characteristic observed in the non-cultured tissue under similar conditions. For example, a cultured hippocampal tissue or other brain tissue is considered to have a natural architectural characteristic if the architecture of the cultured hippocampal tissue is similar to the architecture of hippocampal tissue that has not undergone a culture process (such as an acute hippocampal slice) using similar visualization techniques. In yet another example, a cultured hippocampal or other brain tissue is considered to have a natural neuronal circuit function if the neuronal circuit function of the cultured hippocampal tissue is similar to the neuronal circuit function of hippocampal tissue that has not undergone a culture process (such as an acute hippocampal slice) using similar experimental techniques.

As a result, the current method provides a means to study morphological and physiological events over time. Because the tissue slices are retained in their natural architecture and organization, the cells comprising the tissue slices are able to retain their natural function and relationship to other cells. For example, hippocampal slices obtained from rat brains and cultured for 10 days under the SMC as described retain electrophysiological properties similar to acute hippocampal slices obtained from control animals. In addition, because natural tissue architecture is retained, the complex neuronal circuits are retained, allowing for more accurate physiological studies. This maintenance of natural architecture also allows for population recordings to be obtained. The present disclosure shows that tissue slices grown under the SMC conditions disclosed remain viable and retain natural architecture for at least ten days after the initiation of the culture.

Without being limited to alternate explanations, growing cells under simulated microgravity conditions allows the tissue slices to retain natural architectural and physiological properties by minimizing fluid shear stress, eliminating the high surface tensions created by the air-liquid interface present in 2-dimensional cultures, providing 3-dimensional freedom for cell and substrate spatial orientation and providing increased maintenance of tissue components in a natural spatial organization during the culture process.

Although hippocampal tissue is described below as one application of the current SMC culture method, the method described may be used for any brain tissue of interest. The brain tissue may be isolated from the brainstem region, the diencepalon region, the cerebellum region or the cerebrum region and may comprise any of the various tissues of these regions. This may include brain tissue with natural physiology and pathology and brain tissue with altered physiology and pathology, such as brain tissue from cancerous lesions or brain tissue from epileptic brains. Brain tissue may be obtained from any source. In one embodiment, the brain tissue is obtained from a mammal. In an alternate embodiment, the brain tissue is obtained from a human subject. The brain tissue may be provided as any 3-dimensional tissue section, such as a tissue slice. In addition, certain carrier materials may also be provided for use in the method of the instant disclosure to facilitate 3-dimensional interactions. Brain tissue may also be co-cultured with other types of tissues and cells.

The brain tissues and cells generated by growth under SMC as described may be used for a variety of purposes, including, but not limited to, development of disease models, physiological models and pathological models and for drug screening and drug evaluation. Previous methods for screening and evaluating drugs using cultures of brain tissue were limited, as discussed in more detail above. Furthermore, brain tissues grown under SMC as described exhibit cellular proliferation, suggesting that the method may be used to produce populations of a variety of brain cells/tissues for further study.

The cell culture method described may be used to generate model systems for study of various disease states and physiologic responses. The examples below are meant to be illustrative of the uses of the method disclosed, and should not be considered as limiting the present disclosure in any way. Those skilled in the art may envision alternate models and uses for the method disclosed. In one embodiment, a model of epilepsy may be created by obtaining suitable epileptic brain tissue from an animal or human and culturing that tissue under SMC as described herein or treating non-epileptic tissue with drugs that induce an epileptic state and culturing the tissue under SMC as described herein. The model could be used to screen new therapies that can interfere with epileptic bursting for the prevention/treatment of epilepsy. Following epileptic bursting, axons sprout and form functional connections that are believed to contribute to further increases in epileptic activity. In this way, the model could be used to study axogenesis and synaptogenesis under pathophysiological conditions. Normal brain tissue grown under SMC as described can be made epileptic by increasing the extracellular K⁺ concentration, by blocking GABAA receptors, or by blocking certain types of K⁺ channels. Furthermore, mutant mice (spontaneous mutations) also exist that are genetically predisposed to epileptic bursting. Tissue could be harvested from these animals and grown under SMC conditions as described to test compounds to inhibit seizure activity and to study the development and functional consequences of seizure activity. Finally, mutations in humans have been identified that are known to underlie certain types of epilepsy. Development and prevention of epilepsy could be studied in tissue harvested from transgenic animals carrying the human mutations that cause epilepsy and grown under the SMC conditions described herein. The model system will allow the screening of various drugs to identify new therapeutic compounds for the treatment of epilepsy and to evaluate the mechanism of action of current and future drugs to treat epilepsy in order to elucidate the molecular mechanism of the disease. In one embodiment, a compound to be screened is placed in a culture vessel containing the epileptic brain tissue. The brain tissue is cultured in the presence of said compound under microgravity conditions. After an appropriate incubation time, a brain tissue specimen is removed and the effect of the compound is determined on a characteristic associated with the epileptic state (such as a physiological response, for example effect of the compound on the generation of EPSP, which is discussed in more detail below). The method for determining the effect of the compound on any given characteristic associated with the epileptic state will vary depending on the characteristic. Methods for such determinations are well-known in the art. In one embodiment, the effect of the compound is determined by comparing the relevant characteristic from brain tissue culture in the presence of the compound with brain tissue cultured in the absence of the compound.

In an alternate embodiment, a model of neuroprotection may be created by obtaining brain tissue from an animal or human and culturing that tissue under SMC as described herein. The method disclosed may be used to test new therapies that will protect neurons from death resulting from hypoxia (as occurs in stroke), loss of growth factors, loss of endocrine factors (such as, but not limited to, loss of estrogen following menopause), ion channel mutations, neurodegenerative diseases involving polyamine repeats, to name a few. As one example, a model of hypoxia may be generated to study the process of neuronal cell death resulting from hypoxia. Such a model would have applications to the study of strokes and other conditions where ischemic conditions are generated. In one embodiment, a hypoxic culture is created by treating the culture system containing the tissue with N₂; other methods for creating hypoxic conditions are well-known known in the art. As discussed above, such a model will allow the testing of various drugs to screen new therapeutic compounds for the prevention, amelioration and treatment of neuronal cell death resulting from hypoxia and to evaluate the mechanism of action of current and future drugs in order to elucidate the molecular mechanism of action. Such compounds may be evaluated as discussed above.

In yet another alternate embodiment, a model of neurogenesis may be created by obtaining brain tissue from an animal or human and culturing that tissue under SMC as described herein. Recent studies show that neurogenesis can occur in the adult nervous system (particularly in the hippocampus) following several types of stimuli, both physiological and pathological. The tissue culture system described herein could be used to test compounds that either enhance or prevent neurogenesis. Such compounds may be evaluated as discussed above.

In still another alternate embodiment, a model of brain cancer, such as glioma, may be created by obtaining brain tissue from an animal treated to induce such cancers or human subject suffering from such cancers and culturing that tissue under SMC as described herein. In this model, cancer cells (such as, but not limited to, gliomas) are implanted into the brain of a test animal at a desired location and allowed to develop into a solid tumor. Following tumor development, brain tissues, such as slices, containing the tumor will be prepared and cultured under SMC as described. The model will permit the testing of potential anticancer therapies that will target and destroy tumor cells without harming the surrounding neurons and normal glia cells. This model will also be useful for studying cancer metastasis and identify and develop agents that will inhibit metastasis. Such a model will increase our understanding of the mechanisms that tumor cells use to grow and invade healthy brain tissue. Such information will allow for the development of new therapeutic strategies for the treatment and management of brain tumors.

As used in this specification, the term microgravity means any environment in which the apparent weight of an object or system is small compared to its actual weight on earth due to gravity (which is defined as 1 G). Simulated microgravity conditions means any environment used to simulate microgravity in a 1 G environment; SMC will be understood to mean the tissue of interest is grown in a three-dimensional environment.

### Simulated Microgravity Cell (SMC) Culture System

The operation of Synthecon's RCCS™ is a culture system designed to simulate microgravity conditions in a 1 G environment. The RCCS™ system is the subject of several U.S. Patents, including U.S. Pat. Nos. 4,998,632, 5,026,650 and 5,115,034 (which are each incorporated by reference herein). The principles of operation of the RCCS™ are described in detail in the referenced patent applications. The system provides minimized mechanical stresses, such as fluid shear stress and surface tension stress, increased 3-D freedom for cell and substrate spatial orientation and increased maintenance of tissue components in a natural spatial organization during the culture process. Although the RCCS system is described in detail below, any culture system that provides these or similar properties may be used in the practice of the instant disclosure. Briefly, the cylindrical culture vessel is filled with culture medium and the tissues of interest are added. All air bubbles are removed from the culture vessel. Air bubbles may cause disruption of fluid streamlines and subject the culture to adverse shear effects which may negatively impact growth and/or viability of the cultures. The vessel is attached to a rotator base and rotated about the horizontal axis. The cells or tissues establish a fluid orbit within the culture medium in the horizontally rotating cylindrical vessel and do not contact the walls of the vessel or any other parts of the vessel, and may appear as if embedded in gelatin.

As tissues grow in size the rotation speed of the culture vessel is adjusted to compensate for the increased settling rates of the larger particles. The tissues do move enough within the fluid culture medium to exchange nutrients, wastes and dissolved gases. Oxygen supply and carbon dioxide removal is achieved through a gas permeable silicone rubber membrane. Since the RCCS™ has no impellers, airlifts, bubbles, or agitators to cause tissue damage from impact and turbulence, stress and damage to the tissue is significantly decreased as compared to conventional bioreactors. Shear stress and damage is so low that it is essentially insignificant. Under these conditions, cells and tissues are allowed to grow in a simulated natural environment.

Unlike tissue cultures grown in two-dimensional flat plate systems, tissues grown in the RCCS™ are functionally similar to tissues found in the human body. As a result, cells and tissues can be grown in vitro that mimic the structure and function of the same tissue in vivo. This is especially important in the testing and evaluation of drug candidates and the creation and establishment of relevant models systems for the studies of disease states and physiological responses.

### Hippocampal Cells Grown Under Simulated Microgravity Conditions Exhibit Natural Cellular Organization

The hippocampus is organized into complex cell layers. This specific organization allows the development of natural electrical properties which sustain learning and memory. The tissue architecture and organization of hippocampal tissues grown under SMC as described herein was examined. Hippocampal cultures were prepared from Sprauge-Dawley rats as described. Control hippocampal tissue was also prepared for comparison purposes.

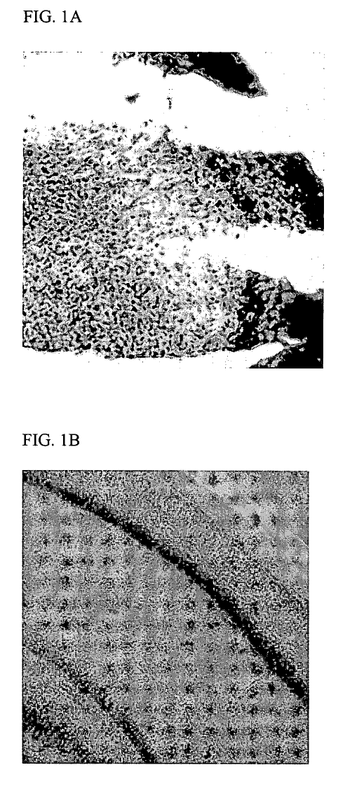

The hippocampal tissue was cultured in the Synthecon RCCS™ for 10 days in Natural Culture Medium under the conditions described. After 10 days in culture, the hippocampal cultures were assayed by Niss'l staining and compared to control hippocampal tissue taken from the original brain tissue used to initiate the hippocampal cell culture. The results are shown in FIGS. 1A and 1B. FIG. 1A shows Niss'l staining from hippocampal brain tissue grown under SMC and FIG. 1B showing Niss'l staining from control hippocampal tissue. As can be seen in FIG. 1A, hippocampal cultures grown under SMC exhibited the same cellular organization as control hippocampal tissue, showing a distinct pyramidal cell layer and striatum radiatum layer. These results show that brain tissues, and specifically hippocampal cultures, grown under SMC are able to proliferate and maintain their structural organization as multi-cellular layers

### Hippocampal Cell Grown Under Simulated Microgravity Conditions Have Intact Cell Membranes.

In order to determine the viability of hippocampal cultures grown under SMC, a viability assay was performed. Hippocampal tissue cultures were prepared and grown in the Synthecon RCCS™ for 10 days in Normal Culture Medium as described. After 10 days in culture without any change of media, the slices were assayed for viability using a LIVE/DEAD Viability/Cytotoxicity assay (Molecular Probes, assay completed as per manufacturers instructions). This assay takes advantage of differential permeability of live and dead cells to a pair of fluorescent stains, ethidium homodimer-1 and calcein AM. Ethidium homodimer-1 (a nucleic acid stain) is impermeable to intact cellular membranes, and requires disrupted membranes (i.e., non-viable cells) for detectable staining. Cells with compromised membranes (non-viable cells) exhibit red-fluorescence from ethidium homodimer-1 staining of nucleic acid. The non-fluorescent calcein AM is freely permeable to intact cell membranes. Inside viable cells, calcein AM is converted by non-specific cytosolic esterases to the bright green-fluorescent calcein. Viable cells exhibit green-fluorescence as a result of calcein staining.

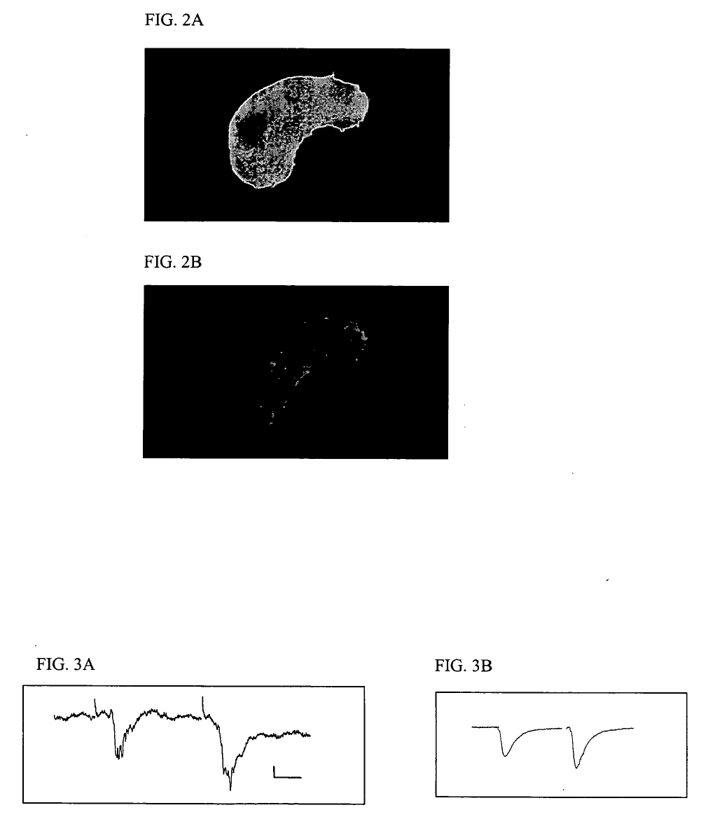

FIGS. 2A and 2B show the results of the viability assay on 10 day old hippocampal cultures grown under SMC. FIG. 2A shows calcein staining of viable hippocampal cells and FIG. 2B shows ethidium homodimer-1 staining of non-viable hippocampal cells. As is evident, the majority of cells from hippocampal cultures grown under SMC have intact cell membranes (FIG. 2A). Some cytotoxicity is also visible (FIG. 2B), which may be due to tissue necrosis in the periphery at the site where recording and stimulating electrodes were implanted for electrophysiological studies (described below).

### Electrical Stimulation Evokes Excitatory Postsynaiptic Potential in Hippocampal Tissues Grown Under Simulated Microgravity Conditions

In order to determine whether hippocampal tissue grown under SMC is able to generate electrical signals, hippocampal tissue grown under SMC for 10 days as described was electrically stimulated and field excitatory postsynaptic potential (EPSP) recorded. As shown in FIGS. 3A and 3B, hippocampal tissue grown under SMC generated field EPSP's after being stimulated. FIG. 3A shows field EPSP generated in hippocampal tissue grown for 10 days under SMC, and FIG. 3B shows a field EPSP in a control hippocampal brain slice taken from the original brain tissue used to generate the hippocampal tissue culture. One important characteristic of the response is the presence of paired-pulse facilitation (PPF) (FIGS. 3A and 3B). PDF have been described and characterized in acute slices from the hippocampus and are an example of frequency-dependent short-term plasticity of excitatory synapses. This phenomenon is accounted for by the residual calcium hypothesis, according to which the small fraction of calcium entering the terminal during the first spike increases the probability of transmitter release to a second action potential (Zucker, 1989). It follows that if repetitive stimulation of presynaptic neurons causes a reduction in the release probability and, therefore, in the amplitude of the first EPSC (Thomson et al. 1993), it should induce a further facilitation of the second EPSC.

The results shown in FIGS. 3A and 3B show that brain tissue, in this example hippocampal tissue, grown under SMC showed EPSP in response to electrical stimulation similar to that observed in control hippocampal slices of the same post-natal period. Moreover, it was possible to generate PPF indicating the presence of short-term plasticity in this tissue.

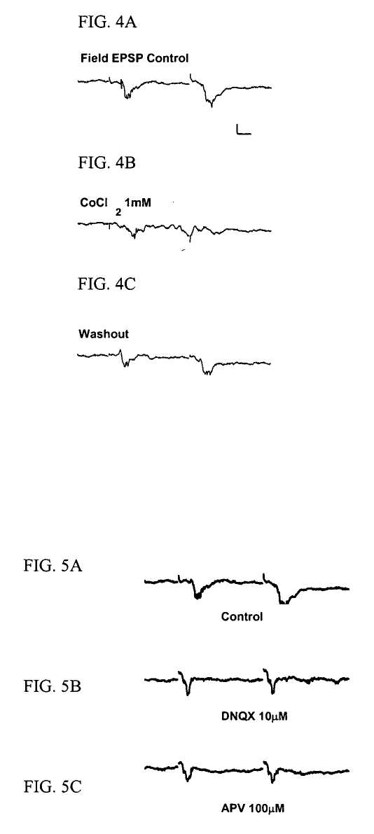

In order to determine if the above field EPSP responses were dependent on the entry of calcium via voltage gated calcium current, the hippocampal tissue grown under SMC (10 days) as described was perfused with 1 mM of cobalt chloride (CoCl₂). As shown in FIG. 4B, 1 mM CoCl₂ reversibly blocked both EPSP observed in hippocampal cells stimulated in the absence of CoCl₂ (FIG. 4A). The effect of CoCl₂ was reversible after washout of CoCl₂ in normal culture medium (FIG. 4C). The results shown in FIGS. 4A-C demonstrate that brain tissue grown under SMC showed the expected inhibition of field EPSP when voltage-gated calcium currents were inhibited.

In addition to inhibition by CoCl₂, field EPSP are also inhibited by glutamatergic antagonists. Glutamate is the major excitatory transmitter of the central nervous system, and in particular the hippocampus. Glutamate interacts with receptors which can be divided into two major classes, the non-NMDA (N-methyl-D-aspartate) and the NMDA receptors. Both the NMDA and non-NMDA receptors are ligand-activated channels. Once activated by the binding of glutamate to the specific receptors, these channels open and allow a cascade of cellular events that when integrated underlie cognitive functions, such as learning and memory.

EPSPs are mediated by α-amino-3-hydroxy-5-methylisoxazole-4-propionate (AMPA) subtype (Collingridge et al 1983) and the NMDA subtype of glutamate receptors. FIGS. 5A-C show the effect of glutamatergic antagonists on field EPSP in hippocampal cells cultured under SMC. Hippocampal tissues were perfused with specific antagonists for these glutamatergic receptors, DNQX (7-nitro-quinoxaline-2,3-dione, which inhibits the AMPA subtype of glutamate receptors) and APV (amino-5-phosphonovaleric acid, which inhibits the NMDA subtype of glutamate receptors) respectively. As shown, either 10 μM DNQX (FIG. 5B) or 100 μM APV (FIG. 5C) are able to partially block field EPSPs evoked in hippocampal tissues grown under SMC as compared to hippocampal tissues grown under SMC and not exposed to these antagonists (FIG. 5A). This important result indicated that NMDA and non-NMDA components are involved in the excitatory neurotransmission in the hippocampus cultured under microgravity condition. These results indicate that the hippocampal slices grown under SMC retain natural 3-D architecture and can respond to well characterized stimuli and antagonists in the same manner as hippocampal tissues studies under acute conditions.

### Analysis of Protein Expression in Hippocampal Tissue Grown Under Simulated Microgravity Conditions

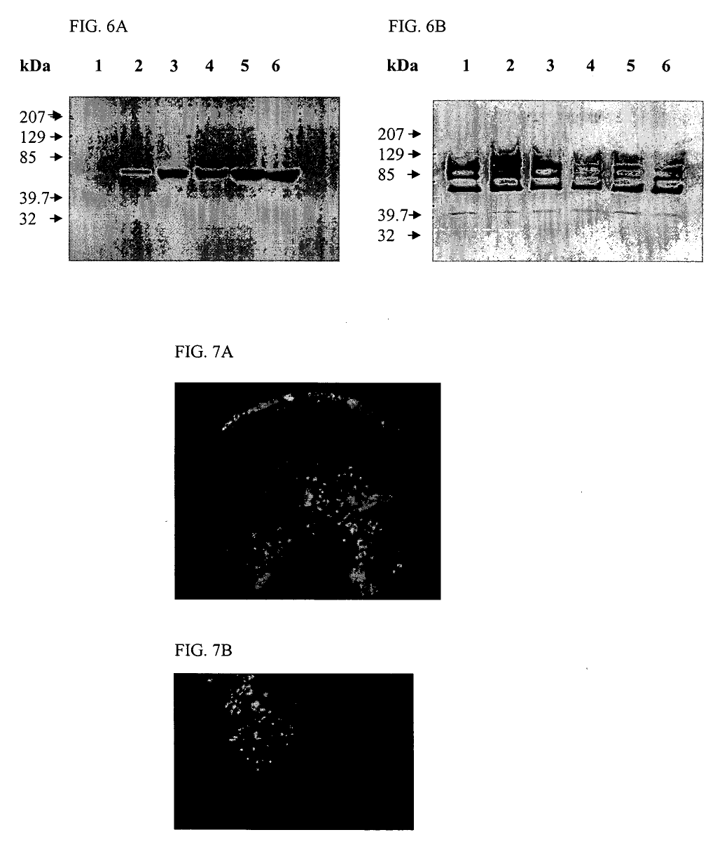

The protein expression of brain tissue grown under SMC was examined. Protein from whole cell lysates was prepared from acute hippocampal brain slices and analyzed by Western blotting as described below. FIGS. 6A and B show the results of Western blots analyzing glycine receptor (GlyR) and glial fibrillary acidic protein (GFAP) expression. GFAP is specific to astrocytes and GlyR is specific to pyramidal neurons and interneurons. As shown in FIGS. 6A and 6B, GlyR and GFAP were detected in all the acute hippocampal slices

In order to study the presence and localization of proteins of interest in the hippocampal tissue grown under SMC, immuno-staining was performed using anti-GFAP monoclonal antibody. GFAP immunoreactivity and mRNA expression have been shown to be useful neurodegenerative markers. For example, GFAP expression is increased in response to injury, neurodegenerative disease and aging (Gross and Morgan 1995; Linnemann and Skarsfelt 1994). The increased expression of GFAP corresponds to cellular hypertrophy, referred to as astrogliosis (Eng 1985; Lindsay 1986). In a recent study, Day et al (1998) demonstrated that 14-day exposure to microgravity reduces GFAP mRNA expression, but does not significantly alter the level of GFAP protein expressions in hippocampal astrocytes.

FIGS. 7A and 7B show that GFAP protein is highly expressed in a hippocampal cells grown under SMC. In addition, GFAP exhibits a similar distribution pattern in hippocampal tissue grown under SMC as seen in acute hippocampal slices.

All references to articles, books, patents, websites and other publications in this disclosure are considered incorporated by reference.

### METHODS

### Western Blotting

Rat brains were removed and sliced with a vibratome to approximately 400 μm. The hippocampal structure was dissected out. Whole cell lysate of the hippocampal structure was prepared by homogenation in PBS (pH7.4) containing 0.2% Triton X-100, using a Teflon-glass homogenizer. The homogenate was centrifuged (11,000 g, 10 min) and aliquots of the supernatant used to determine protein concentration (Bradford 1976). Western blots (Sambrook and Gething 1989) were performed using equal quantities of protein separated on 4-20% Tris-glycine polyacrylamide gel electrophoresis (PAGE) precast gels (BioRad) with stained molecular markers (Kaleidoscope, BioRad) loaded for reference. Proteins were then electrophoretically transferred according to the methods of Towbin et al. (1979) onto PVDF membranes (Amersham). The membranes were blocked at room temperature (RT, 1 h) with dried milk (5%) in Tris-buffered saline with Tween (TBS-T), washed briefly in TBS-T and incubated (4° C., overnight) with appropriate antibodies. GFAP was detected with MAb36 (Chemicon, USA) and GlyR was detected mAb 4a IgG1 (Connex GmbH, Germany). Membranes were washed with TBS-T (RT, 30 min), and then incubated (RT, 1 hr) with horseradish peroxidase-conjugated goat anti-mouse immunoglobulinG antibody (1:5000, Pierce). Following three 15-min washes with TBS-T, the bands were visualized by Supersignal West Pico chemiluminescent substrate (Pierce) and subject to autoradiography film (Hyperfilm-MP; Amersham) before being developed.

### Preparation of 3-Dimensional Brain Slices

Sprauge-Dawley rats (7-9 days old) are quickly decapitated and the brain is rapidly removed and placed in ice-cold Gey's solution (g/L): NaCl (7), KCl (0.37), MgSO₄-7H₂O (0.06), MgCl₂-6H₂O (0.21), KH₂PO₄ (0.03), Na₂HPO₄-7H₂O (0.23), Glucose (1), CaCl₂-2H₂O (0.23) and NaHCO₃ (0.027). Coronal slices (400 μm) will be cut immediately from the desired section of the brain. In the instant disclosure, dorsal hippocampus slices were prepared using a vibratome. The appropriate region of the brain will be then isolated from the slices and incubated in cold 1× Gey's Balanced Salt Solution supplemented with 2.77 M D-Glucose and 1 ml 100× pen-strep) and maintained at 4 degree C for 30 minutes. The brain slices will be transferred in 50 ml RCCS chamber (Synthecon) filled with appropriate media. For hippocampal slices, the RCCS chamber is filled with Normal Culture Medium containing: 50% MEM with Earle's salt, without L-glutamine and without phenol red, 25% Earle's Balance Salt Solution with 26mM bicarbonate, 23% define equine serum, 0.5% L-Glutamine, 1.5% D-Glucose, supplemented with 1 ml 100× pen-strep. The rotary speed of the chamber will be adjusted in order to avoid any free fall of the brain slices. The slices will be monitored daily and rotary speed will be adjusted in order to avoid any free falling. After an appropriate period of time in culture, the slices will be processed according to the desired assay. Cell culture media may be replenished as desired.

### Whole Cell Patch Clamp Recordings

Whole cell patch recording are performed using the visual or blind technique (Blanton et al. 1989; Clark and Collingridge 1995). Patch electrodes will be filled with following (in mM): 100 Cs-gluconate or 100 K-gluconate, 0.6 EGTA, 5MgCl₂, 2 ATP-Na₂, 0.3 GTP-Na, and 40 HEPES, pH: 7.2, 260-270 mOsm. Patch electrode resistance will be 4-6 MΩ. Individual slices will be continuously perfused in the recording chamber with normal artificial cerebrospinal fluid (ACSF) containing (in mM): 119 NaCl, 2.5 KCl, 2.5 CaCl₂, 1.3 MgSO₄, 1 NaH₂PO₄, 26 NaHCO₃, 10 glucose and was saturated with 95% 02-5% CO₂ (pH: 7.4). A multiple valve system will be used to perfuse the individual slice with a variety of modified ACSFs containing drugs, including, but not limited to, TTX, CNQX, APV, 4-AP and TEA. In addition neurons will be filled during the recording with 0.4% biocytin for post hoc neuronal identification (McMahon and Kauer 1997 a,b; McMahon et al. 1998). This last procedure is essential because of the thickness of the brain tissue slice, it is impossible to visually identify the recorded cell. Nevertheless, because of different electrical signals between glial cells and neurons, appropriate cells may be identified.

### References

Adams et al. (2000) J Appl Physiol. 88(3):904-16.

Addy (1964) Life Sci Space Res. 2:267-86.

Agmon et al. (1992) J Neurophysiol. 68(1):345-9

Agmon et al. (1993) J Neurosci. 13(12):5365-82

Agmon et al. (1996) J Neurosci. 16(15):4684-95

Bahr et al. (1985)J Comp Neurol. 233(3):405-20.

Belichenko et al. (1988) Biull Eksp Biol Med. 105(6):736-8.

Belichenko et al. (1991) Biull Eksp Biol Med. 112(11):541-2.

Blanton et al. (1989) J Neurosci Methods. December;30(3):203-10.

Bliss et al. Nature. 361(6407):31-9.

Blue et al. (1983) Quantitative analysis. J Neurocytol. (4):697-712.

Blue et al. (1983) J Neurocytol. 12(4):599-616.

Bradford (1976) Anal Biochem. 72:248-54.

Buonomano et al. (1998)J Neurophysiol. 80(4):1765-74.

Buonomano et al. (1998) Annu Rev Neurosci. 21:149-86.

Clark et al. (1995) J Physiol. January 1;482 (Pt 1):39-52.

Correia et al. (1992) J Appl Physiol. 73(2 Suppl):112S-120S.

Day et al. (1998) J Appl Physiol. 85(2):716-22.

DeFelipe. (1997) J Chem Neuroanat. 14(1):1-19.

Donoghue. (1995) Curr Opin Neurobiol (6):749-54.

D'yachkova. (1991) Physiologist 34: S185-S186.

D'yachkova et al. (1991) (Abstract). USSR Space Life Sci. Dig. 31: 67.

Eng, (1985) J Neuroimmunol. 1985 June;8(4-6):203-14. Review.

Hubel et al. (1977) Philos Trans R Soc Lond B Biol Sci 278(961):377-409.

Ikemoto et al. (2001) FASEB J. 15(7):1279-81.

Ishihara et al. (1997) Neuroscience. 81(1):275-9

Jones. (2000) J Hist Neurosci. 9 (1):37-9.

Kaas et al. (1983) Annu Rev Neurosci 6:325-56.

Krasnov. (1994) Adv Space Biol Med. 4:85-110.

Linnemann et al. (1994) Neurobiol Aging. September-October;15(5):651-5.

Luhmann et al.(1991) J Neurophysiol.65(2):247-63.

McMahon et al. (1997) J Neurophysiol. November;78(5):2493-502.

McMahon et al. (1997) Neuron. February;18(2):295-305.

McMahon et al. (1998) J Neurosci. August 1;18(15):5640-51.

Micheva et al. (1996) J Comp Neurol. 373(3):340-54.

Miller et al. (1986) J Neurosci. January;6(1):22-9.

Newberg et al. (1998) Adv Space Res. 22(2):185-96.

Rampon et al.(2000) Nat Neurosci. 3(3):238-44.

Robertson et al. (1994) Am J Med Sci. 308(2):126-32.

Ross. (1994) Acta Otolaryngol Suppl. 516:1-14

Roy et al. (1996) Exerc Sport Sci Rev. 24:399-425.

Sambrook et al (1989) Nature. 342 (6247):224-5.

Shimizu et al. (2000) Science. 290(5494): 1170-4

Suret et al. (2001) Nat Rev Neurosci. 2 (4):251-62.

Thomson et al. (1993) J Neurophysiol. 70(6):2354-69

Towbin et al. (1979). Proc Natl Acad Sci U S A. 76 (9):4350-4.

Tsien et al. (1996) Cell. 87(7):1327-38.

Van der Loos et al. (1973) Science 179 (71):395-8.

Walton et al. (1992) Neuroscience. 51(4):763-7.

Wells et al. (2000) Curr Opin Neurobiol. 10(1):132-7.

Wells et al. (2000) J Neurosci. 20(23):8822-30

White et al. (1997) Cereb Cortex. 7(1):31-47.

White et al. (1997) Cereb Cortex. 7(1):18-30

Zucker. (1989Annu Rev Neurosci. 12:13-31.

What is claimed:

1. A method of culturing a three-dimensional section of brain tissue, said method comprising the steps of
a. isolating said brain tissue;
b. placing said isolated brain tissue into a culture vessel containing a suitable culture media;
c. culturing said brain tissue under microgravity conditions whereby said brain tissue retains a natural tissue characteristic.

2. The method of claim 1 where said natural tissue characteristic is selected from the group consisting of: (i) tissue architecture, (ii) tissue organization, (iii) physiological responses to certain stimuli, (iv) physiological response to agonists and inhibitors, (v) electrophysiological responses, (vi) maintenance of neuronal circuits, and (vii) a combination of any of the foregoing.

3. The method of claim 1 where said brain tissue is derived from a brainstem region, a diencepalon region, a cerebellum region a cerebrum region.

4. The method of claim 1 where said brain tissue is selected from the group consisting of: a hippocampal tissue, a thalamus tissue, a hypothalmus tissue, an epithalmus tissue, a basal ganglia tissue, a cortex tissue, a corpus collosum tissue and an amygdale tissue.

5. The method of claim 1 where said tissue is a hippocampal tissue.

6. The method of claim 5 where said hippocampal tissue is a tissue slice.

7. The method of claim 1 where said microgravity conditions are simulated microgravity conditions.

8. The method of claim 1 where said simulated microgravity conditions comprise at least one of the following characteristics: low fluid shear stress, reduced surface tension as compared to 2-dimenional culture systems, 3-dimensional freedom for cell and substrate spatial orientation, and providing increased maintenance of tissue components in a natural spatial organization during the culture process.

9. The method of claim 1 where said brain tissue remains viable for a period of at least 10 days.

10. The method of claim 1 where said brain tissue exhibits natural response to electrophysiological stimuli.

11. The method of claim 1 where said brain tissue maintains natural neuronal circuitry.

12. A method for maintaining a natural neuronal circuit function in a culture of a three-dimensional section of brain tissue, said method comprising the steps of:
a. isolating said brain tissue with a natural neuronal circuit function;
b. placing said isolated brain tissue into a culture vessel containing a suitable culture media;
c. culturing said brain tissue under microgravity conditions whereby said natural neuronal circuit function is maintained.

13. The method of claim 12 where said brain tissue is derived from a brainstem region, a diencepalon region, a cerebellum region a cerebrum region.

14. The method of claim 12 where said brain tissue is selected from the group consisting of: a hippocampal tissue, a thalamus tissue, a hypothalmus tissue, an epithalmus tissue, a basal ganglia tissue, a cortex tissue, a corpus collosum tissue and an amygdale tissue.

15. The method of claim 12 where said tissue is a hippocampal tissue.

16. The method of claim 15 where said hippocampal tissue is a tissue slice.

17. The method of claim 12 where said microgravity conditions are simulated microgravity conditions.

18. The method of claim 12 where said simulated microgravity conditions comprise at least one of the following characteristics: low fluid shear stress, reduced surface tension as compared to 2-dimenional culture systems, 3-dimensional freedom for cell and substrate spatial orientation, and providing increased maintenance of tissue components in a natural spatial organization during the culture process.

19. The method of claim 12 where said brain tissue remains viable for a period of at least 10 days.

20. The method of claim 12 where said brain tissue exhibits natural response to electrophysiological stimuli.

21. The method for isolating compounds that are effective against epilepsy, said method comprising the steps of:
a. isolating a brain tissue from an epileptic subject;
b. placing said isolated brain tissue into a culture vessel containing a suitable culture media;
c. placing a compound in said culture vessel;
d. culturing said brain tissue in the presence of said compound under microgravity conditions whereby said brain tissue retains a natural tissue characteristic associated with an epileptic state; and
e. determining the effect of said compound on at least one of said natural tissue characteristic associated with said epileptic state.

22. The method of claim 21 where the subject is a mammal.

23. The method of claim 21 where the subject is a human.

24. The method of claim 21 where said natural tissue characteristic associated with the epileptic state is selected from the group consisting of: (i) tissue architecture, (ii) tissue organization, (iii) physiological responses to certain stimuli, (iv) physiological response to agonists and inhibitors, (v) electrophysiological responses, (vi) maintenance of neuronal circuits, and (vii) a combination of any of the foregoing.

25. The method of claim 21 where said brain tissue is derived from a brainstem region, a diencepalon region, a cerebellum region a cerebrum region.

26. The method of claim 21 where said tissue is a hippocampal tissue.

27. The method of claim 26 where said hippocampal tissue is a tissue slice.

28. The method of claim 21 where said microgravity conditions are simulated microgravity conditions.

29. The method of claim 28 where said simulated microgravity conditions comprise at least one of the following characteristics: low fluid shear stress, reduced surface tension as compared to 2-dimenional culture systems, 3-dimensional freedom for cell and substrate spatial orientation, and providing increased maintenance of tissue components in a natural spatial organization during the culture process.
